(19) United States
(12) Patent Application Publication
(10) Pub. No.: US 2025/0281759 A1
(43) Pub. Date: Sep. 11, 2025

(54) PROGRAMMING AND PACING THERAPY OPTIMIZATION

(71) Applicant: Cardiac Pacemakers, Inc., St. Paul, MN (US)

(72) Inventors: Ramin Zandvakili, Tehran (IR); Mojgan Goftari, Shoreview, MN (US); Pramodsingh Hirasingh Thakur, Woodbury, MN (US)

(21) Appl. No.: 19/062,359

(22) Filed: Feb. 25, 2025

Related U.S. Application Data
(60) Provisional application No. 63/561,428, filed on Mar. 5, 2024.

(51) Int. Cl. A61N 1/37; A61N 1/365; A61N 1/372; G16H 10/60; G16H 20/40; G16H 50/70

(52) U.S. Cl. CPC A61N 1/371; A61N 1/365; A61N 1/37247; G16H 10/60; G16H 20/40; G16H 50/70

(57) ABSTRACT

Systems and methods to improve programming of medical devices and delivery of cardiac pacing are disclosed, including receiving parameter settings of an ambulatory medical device, processing the received parameter settings by inputting the received parameter settings into one or more pre-trained machine learning models to identify one or more differences between the parameter settings of the ambulatory medical device and the model parameter settings of one or more other ambulatory medical devices, and generating a programming recommendation for the ambulatory medical device to improve cardiac capture for the patient based on the identified one or more differences.

### CLAIM OF PRIORITY

This application claims the benefit of U.S. Provisional Application No. 63/561,428, filed on Mar. 5, 2024, which is hereby incorporated by reference in its entirety.

### TECHNICAL FIELD

This document relates generally to programming medical devices, and more particularly, but not by way of limitation, to systems and methods to optimize programming of medical devices to improve pacing therapy.

### BACKGROUND

Heart failure (HF) is a reduction in the ability of the heart to deliver enough blood to meet bodily needs. Heart failure patients commonly have enlarged hearts with weakened cardiac muscles, resulting in reduced contractility and poor cardiac output. Signs of heart failure include pulmonary congestion, edema, difficulty breathing, etc. Heart failure is often a chronic condition, but can also occur suddenly, affecting the left, right, or both sides of the heart. Causes of heart failure include coronary artery disease, myocardial infarction, high blood pressure, atrial fibrillation, valvular heart disease, alcoholism, infection, cardiomyopathy, or one or more other conditions leading to a decreased pumping efficiency of the heart.

Medical devices, including ambulatory, implantable, subcutaneous, wearable, or one or more other medical devices, etc., can monitor, detect, or treat various conditions including heart failure, atrial fibrillation, etc. Medical devices can include sensors to obtain or sense physiologic information from a patient and one or more circuits to detect one or more physiologic events using the sensed physiologic information or transmit sensed physiologic information or detected physiologic events to one or more remote devices. Additionally, medical devices can be configured to provide electrical stimulation or one or more other therapies or treatments to the patient, such as to improve cardiac function, etc.

### SUMMARY

Systems and methods to improve programming of medical devices and delivery of cardiac pacing are disclosed, including receiving parameter settings of an ambulatory medical device, processing the received parameter settings by inputting the received parameter settings into one or more pre-trained machine learning models to identify one or more differences between the parameter settings of the ambulatory medical device and the model parameter settings of one or more other ambulatory medical devices, and generating a programming recommendation for the ambulatory medical device to improve cardiac capture for the patient based on the identified one or more differences.

An example of subject matter (e.g., a computing device or a medical device system, such as for generating a programming recommendation for an ambulatory medical device to improve cardiac capture in a patient during cardiac resynchronization therapy by the ambulatory medical device) may comprise means for receiving parameter settings of an ambulatory medical device, means for processing the received parameter settings by inputting the received parameter settings into one or more pre-trained machine learning models, each of the one or more pre-trained machine learning models trained to compare the received parameter settings to stored model parameter settings from one or more other ambulatory medical devices corresponding to one or more other patients and to identify one or more differences between the parameter settings of the ambulatory medical device and the stored model parameter settings of the one or more other ambulatory medical devices, and means for upon obtaining an output from the one or more pre-trained machine learning models indicating the identified one or more differences between the parameter settings of the ambulatory medical device and parameter settings of the one or more other ambulatory medical devices, generating the programming recommendation for the ambulatory medical device to improve cardiac capture for the patient based on the identified one or more differences.

In an example, which may be combined with any one or more examples described herein, the means for receiving, the means for processing, and the means for obtaining comprise one or more processors and one or more memory devices storing instructions, which when executed by the processor, cause the one or more processors to perform operations comprising receiving parameter settings of the ambulatory medical device, processing the received parameter settings by inputting the received parameter settings into one or more pre-trained machine learning models, each of the one or more pre-trained machine learning models trained to compare the received parameter settings to stored model parameter settings from one or more other ambulatory medical devices corresponding to one or more other patients and to identify one or more differences between the parameter settings of the ambulatory medical device and the stored model parameter settings of the one or more other ambulatory medical devices, and upon obtaining an output from the one or more pre-trained machine learning models indicating the identified one or more differences between the parameter settings of the ambulatory medical device and parameter settings of the one or more other ambulatory medical devices, generating the programming recommendation for the ambulatory medical device to improve cardiac capture for the patient based on the identified one or more differences.

In an example, which may be combined with any one or more examples described herein, to identify the one or more differences between the parameter settings of the ambulatory medical device and the stored model parameter settings comprises to prioritize the identified one or more differences with respect to reduced cardiac pacing or unsuccessful cardiac capture.

In an example, which may be combined with any one or more examples described herein, the operations may further comprise receiving cardiac capture information of the patient during cardiac resynchronization therapy delivered by the ambulatory medical device according to the received parameter settings, wherein processing the received parameter settings further comprises inputting the received cardiac capture information of the patient into the one or more pre-trained machine learning models, each of the one or more pre-trained machine learning models trained to compare the received parameter settings and the received cardiac capture information to stored model parameter settings and stored model cardiac capture information from one or more other ambulatory medical devices corresponding to one or more other patients and to identify one or more differences between the parameter settings of the ambulatory medical device and the stored model parameter settings of the one or more other ambulatory medical devices, wherein generating the programming recommendations comprises generating a reprogramming recommendation for the ambulatory medical device to optimize cardiac capture for the patient, wherein the ambulatory medical device comprises an implantable cardiac resynchronization therapy device implanted in the patient.

In an example, which may be combined with any one or more examples described herein, the operations may further comprise receiving physiologic information of the patient obtained by the ambulatory medical device and determining an indication of cardiac capture of the patient during cardiac resynchronization therapy delivered by the ambulatory medical device according to the received parameter settings using the received physiologic information.

In an example, which may be combined with any one or more examples described herein, the operations may further comprise receiving physiologic information of the patient obtained by the ambulatory medical device, wherein processing the received parameter settings further comprises inputting the received physiologic information of the patient into the one or more pre-trained machine learning models, each of the one or more pre-trained machine learning models trained to compare the received parameter settings and the received physiologic information of the patient to stored model parameter settings from one or more other ambulatory medical devices corresponding to one or more other patients and stored physiologic information from the one or more other patients and to identify one or more differences between the parameter settings of the ambulatory medical device and the stored model parameter settings of the one or more other ambulatory medical devices, wherein generating the programming recommendations comprises to optimize cardiac capture for the patient.

In an example, which may be combined with any one or more examples described herein, the operations further may comprise receiving information about the patient comprising one of demographic information or medical history information separate from sensed physiologic information of the patient, wherein processing the received parameter settings further comprises inputting the received information about the patient into the one or more pre-trained machine learning models, each of the one or more pre-trained machine learning models trained to compare the received parameter settings and the received physiologic information of the patient to stored model parameter settings from one or more other ambulatory medical devices corresponding to one or more other patients and stored information about the one or more other patients and to identify one or more differences between the parameter settings of the ambulatory medical device and the stored model parameter settings of the one or more other ambulatory medical devices, wherein generating the programming recommendations comprises to optimize cardiac capture for the patient.

In an example, which may be combined with any one or more examples described herein, the operations may further comprise providing the generated programming recommendation to a user or process.

In an example, which may be combined with any one or more examples described herein, providing the generated programming recommendation to the user or process includes providing an output of the generated programming recommendation to a user interface for display to the user or to a control circuit to control or adjust the process or function of the ambulatory medical device.

In an example, which may be combined with any one or more examples described herein, the operations may further comprise reprogramming the ambulatory medical device using the generated programming recommendation including changes to one or more parameter settings and providing cardiac resynchronization therapy to the patient according to the one or more reprogrammed parameter settings.

In an example, which may be combined with any one or more examples described herein, generating the programming recommendation for the ambulatory medical device to improve cardiac capture for the patient based on the identified one or more differences includes implementing at least one of a set of rules associated with the following parameter settings Atrioventricular Delay Fixed and Atrioventricular Dynamic Maximum.

In an example, which may be combined with any one or more examples described herein, generating the programming recommendation for the ambulatory medical device to improve cardiac capture for the patient based on the identified one or more differences includes implementing at least one of a set of rules associated with at least two of the following parameter settings Atrial Tachy Response Mode, Biventricular Trigger Enable, Ventricular Tachycardia Zone Rate, Atrial Tachy Response Trigger Rate, Maximum Sensor Rate Interval, Ventricular Tachycardia 1 Zone Rate, Number of Ventricular Zones, Ventricular Fibrillation Zone Rate, Atrial Tachy Response Ventricular Rate Regulation Response, Atrial Tachy Response Biventricular Trigger Enable, Atrial Tachy Response Lower Rate Limit, Tachycardia Mode, Respiration Rate Trend Enable, Atrial Tachy Response Pacing Chamber, and Sensing Mode.

In an example, which may be combined with any one or more examples described herein, generating the programming recommendation for the ambulatory medical device to improve cardiac capture for the patient based on the identified one or more differences includes implementing at least one of a set of rules associated with at least two of the following parameter settings Sensed Atrioventricular Delay, Atrioventricular Dynamic Minimum, Atrioventricular Delay Fixed, and Atrioventricular Dynamic Maximum.

An example of subject matter (e.g., a computing device or a medical device system, such as for generating a programming recommendation for an ambulatory medical device to improve cardiac capture in a patient during cardiac resynchronization therapy by the ambulatory medical device) may comprise one or more processors and one or more memory devices storing instructions, which when executed by the processor, cause the one or more processors to perform operations comprising receiving physiologic information of the patient obtained by the ambulatory medical device, receiving parameter settings of the ambulatory medical device, receiving cardiac capture information of the patient during cardiac resynchronization therapy delivered by the ambulatory medical device according to the received parameter settings, upon receiving or determining an indication of a loss of cardiac capture of a heart using the received physiologic information of the patient obtained by the ambulatory medical device, processing the received parameter settings by inputting the received parameter settings into one or more pre-trained machine learning models, each of the one or more pre-trained machine learning models trained to compare the received parameter settings to stored model parameter settings from one or more other ambulatory medical devices corresponding to one or more other patients and to identify one or more differences between the parameter settings of the ambulatory medical device and the stored model parameter settings of the one or more other ambulatory medical devices, and upon obtaining an output from the one or more pre-trained machine learning models indicating the identified one or more differences between the parameter settings of the ambulatory medical device and parameter settings of the one or more other ambulatory medical devices, generating the programming recommendation for the ambulatory medical device based on the identified one or more differences.

In an example, which may be combined with any one or more examples described herein, the operations may further comprise upon obtaining an output from the one or more pre-trained machine learning models indicating differences between the parameter settings of the ambulatory medical device and parameter settings of the one or more other ambulatory medical devices, prioritizing the one or more differences with respect to a potential or detected loss of cardiac capture or reduced pacing.

An example of subject matter (e.g., a method, such as for generating a programming recommendation for an ambulatory medical device to improve cardiac capture in a patient during cardiac resynchronization therapy by the ambulatory medical device) may comprise receiving, over a network, parameter settings of the ambulatory medical device, processing, using one or more processors, the received parameter settings by inputting the received parameter settings into one or more pre-trained machine learning models, each of the one or more pre-trained machine learning models trained to compare the received parameter settings to stored model parameter settings from one or more other ambulatory medical devices corresponding to one or more other patients and to identify one or more differences between the parameter settings of the ambulatory medical device and the stored model parameter settings of the one or more other ambulatory medical devices, and upon obtaining an output from the one or more pre-trained machine learning models indicating the identified one or more differences between the parameter settings of the ambulatory medical device and parameter settings of the one or more other ambulatory medical devices, generating, using the one or more processors, the programming recommendation for the ambulatory medical device to improve cardiac capture for the patient based on the identified one or more differences.

In an example, which may be combined with any one or more examples described herein, to identify the one or more differences between the parameter settings of the ambulatory medical device and the stored model parameter settings comprises to prioritize the identified one or more differences with respect to reduced cardiac pacing or unsuccessful cardiac capture.

In an example, which may be combined with any one or more examples described herein, the subject matter may optionally comprise receiving, over the network, cardiac capture information of the patient during cardiac resynchronization therapy delivered by the ambulatory medical device according to the received parameter settings, wherein processing the received parameter settings further comprises inputting the received cardiac capture information of the patient into the one or more pre-trained machine learning models, each of the one or more pre-trained machine learning models trained to compare the received parameter settings and the received cardiac capture information to stored model parameter settings and stored model cardiac capture information from one or more other ambulatory medical devices corresponding to one or more other patients and to identify one or more differences between the parameter settings of the ambulatory medical device and the stored model parameter settings of the one or more other ambulatory medical devices, wherein generating the programming recommendations comprises generating a reprogramming recommendation for the ambulatory medical device to optimize cardiac capture for the patient, wherein the ambulatory medical device comprises an implantable cardiac resynchronization therapy device implanted in the patient.

In an example, which may be combined with any one or more examples described herein, the subject matter may optionally comprise receiving, over the network, physiologic information of the patient obtained by the ambulatory medical device, and determining, using the one or more processors, an indication of cardiac capture of the patient during cardiac resynchronization therapy delivered by the ambulatory medical device according to the received parameter settings using the received physiologic information.

In an example, which may be combined with any one or more examples described herein, the subject matter may optionally comprise receiving, over the network, physiologic information of the patient obtained by the ambulatory medical device, wherein processing the received parameter settings further comprises inputting the received physiologic information of the patient into the one or more pre-trained machine learning models, each of the one or more pre-trained machine learning models trained to compare the received parameter settings and the received physiologic information of the patient to stored model parameter settings from one or more other ambulatory medical devices corresponding to one or more other patients and stored physiologic information from the one or more other patients and to identify one or more differences between the parameter settings of the ambulatory medical device and the stored model parameter settings of the one or more other ambulatory medical devices, wherein generating the programming recommendations comprises to optimize cardiac capture for the patient.

In an example, which may be combined with any one or more examples described herein, the subject matter may optionally comprise receiving information about the patient comprising one of demographic information or medical history information separate from sensed physiologic information of the patient, wherein processing the received parameter settings further comprises inputting the received information about the patient into the one or more pre-trained machine learning models, each of the one or more pre-trained machine learning models trained to compare the received parameter settings and the received physiologic information of the patient to stored model parameter settings from one or more other ambulatory medical devices corresponding to one or more other patients and stored information about the one or more other patients and to identify one or more differences between the parameter settings of the ambulatory medical device and the stored model parameter settings of the one or more other ambulatory medical devices, wherein generating the programming recommendations comprises to optimize cardiac capture for the patient.

In an example, a system or apparatus may optionally combine any portion or combination of any portion of any one or more of the examples described herein, may optionally combine any portion or combination of any portion of any one or more of the examples described herein to comprise “means for” performing any portion of any one or more of the functions or methods of the examples described herein, or at least one “non-transitory machine-readable medium” including instructions that, when performed by a machine, cause the machine to perform any portion of any one or more of the functions or methods of the examples described herein.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates example rates of cardiac resynchronization therapy (CRT) efficacy.

FIG. 2 illustrates a table of example parameter settings.

FIG. 3 illustrates a diagram of an example machine learning model.

FIG. 4 illustrates a dendrogram of identified hierarchical relationships between different parameter settings.

FIG. 5 illustrates an example method.

FIG. 6 illustrates an example system.

FIG. 7 illustrates an example patient management system and portions of an environment in which the system may operate.

FIG. 8 illustrates an example implantable medical device (IMD) electrically coupled to a heart.

FIG. 9 illustrates a block diagram of an example machine upon which any one or more of the techniques (e.g., methodologies) discussed herein may perform.

### DETAILED DESCRIPTION

Medical devices can be implanted in a patient or otherwise positioned on or about the patient to monitor patient physiologic information, such as heart sound information, respiration information (e.g., respiration rate (RR), tidal volume (TV), rapid shallow breathing index (RSBI), etc.), impedance information (e.g., intrathoracic impedance (ITTI)), pressure information, cardiac electrical information (e.g., heart rate), physical activity information, or other physiologic information or one or more other physiologic parameters of the patient, or to provide electrical stimulation or one or more other therapies or treatments to optimize or control contractions of a heart of the patient. For example, a medical device can include one or more implantable medical devices (IMDs), such as a cardiac resynchronization therapy (CRT) device, etc., configured to receive cardiac electrical information from, and in certain examples, provide electrical stimulation to, one or more electrodes located within, on, or proximate to a heart of the patient, such as coupled to one or more leads and located in one or more chambers of the heart, within the vasculature of the heart near one or more chambers, or otherwise attached to or in contact with the heart.

Cardiac resynchronization therapy generally refers to stimulation therapy generated and provided to one or more chambers of the heart (e.g., frequently two or more of the right ventricle (RV), the left ventricle (LV) (e.g., commonly through the cardiac vasculature), or the right atrium (RA), etc.) to improve cardiac function, such as to improve coordination of contractions between different chambers of the heart (e.g., the right ventricle and the left ventricle, the right atrium and the right ventricle, etc.) or to otherwise improve cardiac output or efficiency. Medical devices can provide different therapies using different therapy modes, however, with different power and resource requirements and varying effectiveness for different patients. A variety of therapy modalities are available to patients, but not all patients receive the optimal medical device, therapy mode, or therapy settings.

The goal of cardiac resynchronization therapy is to effectuate 100% cardiac capture resulting from pacing stimulation, where cardiac capture can refer to LV cardiac capture, RV cardiac capture, or combinations thereof. Recent literature suggests that nearly 40% of current pacing therapy is suboptimal, where suboptimal is defined as cardiac resynchronization therapy resulting in cardiac capture of one or more chambers (typically the LV for CRT patients generally) in less than 98% of cardiac beats. One common reason for suboptimal cardiac resynchronization therapy is inappropriate programming of parameter settings of medical devices (e.g., implantable medical devices) configured to provide cardiac resynchronization therapy (e.g., CRT devices).

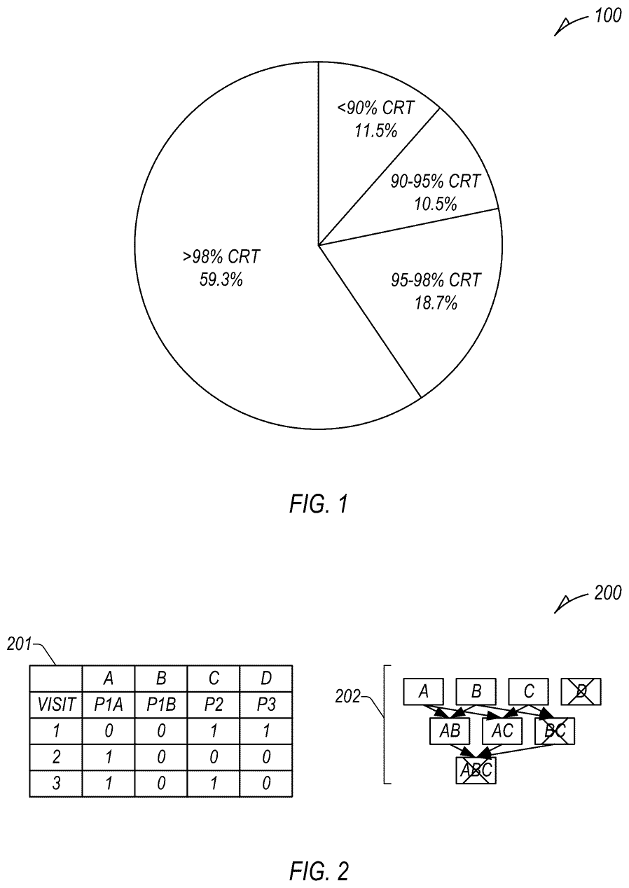

FIG. 1 illustrates example rates of cardiac resynchronization therapy (CRT) efficacy 100 in a third-party study population, including confirmed cardiac capture in greater than 98% of beats in 59.3% of study patients, and three categories of suboptimal cardiac resynchronization therapy: confirmed cardiac capture in 95-98% of beats at 18.7% of study patients; confirmed cardiac capture in 90-95% of beats at 10.5% of study patients; and confirmed capture in less than 90% of beats at 11.5% of study patients.

The present inventors have recognized, among other things, systems and methods to optimize cardiac resynchronization therapy in patients having medical devices (e.g., implantable medical devices) configured to provide cardiac resynchronization therapy (e.g., CRT devices) by analyzing parameter settings and generating reprogramming recommendation for implantable devices to increase optimal pacing, such as by optimizing parameter settings to improve a percentage of confirmed cardiac capture resulting from pacing stimulation. In an example, analyzing parameters can include detecting suboptimal, irregular, or other parameter combinations that may be associated with a suboptimal cardiac capture.

Different pacing parameters can be analyzed using artificial intelligence or machine learning, based on received physiologic information or separate therefrom, to identify optimal and suboptimal combinations of pacing parameters corresponding to successful cardiac resynchronization therapy, such as to optimize pacing parameters to improve rates of cardiac capture resulting from pacing stimulation, including by eliminating or reducing periods of suboptimal, missed, or reduced pacing. Data can be collected and organized for analysis and identification of patterns. Models can be created based on the identified patterns, validated (e.g., using a percentage of confirmed LV cardiac capture, etc.), stored, and deployed. Additionally, deployed models can be monitored and updated as additional data is collected, including retraining as needed.

Medical device systems frequently analyze physiologic information between patients or with respect to one or more clinical thresholds to determine patient condition and optimize device settings. The present inventors have recognized, among other things, that analysis can focus on differences between the parameter settings themselves (e.g., without respect to patient physiologic information, determined indications of cardiac capture or reduced pacing, patient demographics, patient history, etc.), such that a determined similarity between different parameter settings can be analyzed to identify sub-optimal settings or combinations of settings that may result in suboptimal, missed, or reduced pacing. In certain examples, parameter settings can be additionally analyzed with respect to one or more of patient physiologic information (e.g., to identify similar patients, etc.), determined indications of cardiac capture or reduced pacing, patient demographics, patient history, or combinations thereof.

Clinicians have a fair amount of discretion, in determining and implementing parameter settings of medical devices (e.g., CRT devices, etc.), but often follow published literature and guidelines or specific device limits. However, as recommendations change or new therapies, modes, parameters, or settings are introduced, it takes time for such literature or changes in such literature to become widely understood and adopted. For example, certain clinicians may have determined a specific set of parameter settings to optimize pacing in certain patient populations that differ from the previous literature or clinician training. Analysis of settings on a between-patient or between-clinician basis with respect to optimized pacing or capture can identify and determine different combinations of settings and distribute recommended sets of parameter settings more quickly than existing literature. Additionally, whereas clinicians focus on certain parameters, with access to a complete set of parameter settings across large numbers of patients, correlation between seemingly irrelevant parameters in combination with others can be determined that impact cardiac capture rates, improving pacing and cardiac resynchronization therapy, patient outcomes, device performance and efficiency, and communication of leading clinical data more quickly to clinician populations.

Artificial intelligence, particularly machine learning and other techniques, can effectuate the speed and analysis of identifying optimal settings and determining differences between different sets of parameter settings, in combination with physiologic information of the patient (such as determination of patient status, e.g., improving or worsening, etc.) or separate therefrom, taking into account rates of cardiac capture in specific patients or across populations. In addition, separate from tracking rates of cardiac capture for specific patients or patients having specific demographics, disease states, or patient conditions, rates for specific clinicians can be analyzed and determined to identify clinicians having more successful rates of cardiac capture across patients or patient groups.

For example, based on a specific desired output, namely optimizing cardiac resynchronization therapy by effectuating cardiac capture, pacing parameter settings can be analyzed to identify or determine specific parameters or combinations thereof that are more likely correspond to unconfirmed or missed cardiac capture. In an example, although parameter settings often start from a default condition and are separately selectable and adjustable by a clinician, combinations of parameters often ideally move together. In a simple example, if one parameter is adjusted and a second is not, but adjustment of the second often provides optimal cardiac resynchronization therapy, detection of the second not being adjusted can trigger a recommendation to the clinician to adjust the second parameter.

In other examples, such as first programming, or situations where patient physiologic information has not been previously recorded or is otherwise unavailable, proposed parameter settings can be recommended based on other information about the patient, such as age, gender, medications, co-morbidities, diagnosed conditions or disease states or progressions, or other information medical history information separate from sensed physiologic information. In this way, the first programmed values for a specific patient can differ from default values for all patients, potentially improving the speed of attaining optimal programming and reducing wasted resources associated with suboptimal operation.

Additionally, optimal parameter settings, or suggested combinations of parameter settings to optimize cardiac resynchronization therapy, may adjust over time, just as the parameters and settings themselves. In certain examples, optimal combinations of parameter settings or suggestions to optimize parameters settings for a particular patient can be provided to a clinician, such as during follow-up with the patient, even in the absence of unconfirmed or missed cardiac capture. In other examples, suboptimal pacing, including unconfirmed or confirmed missed cardiac capture, can trigger analysis and recommendation.

Periods of suboptimal pacing can be harmful to patients but may also lead to inefficient use of device resources including periods of stimulation by the device that may not provide a desired physiologic response, effectively wasting limited device resources. Identifying potentially less effective or ineffective parameter settings or combinations of parameter settings and providing a recommendation of one or more programming changes to improve pacing can result in a more efficient use of device resources while also improving patient therapy. Accordingly, identification of suboptimal settings and generating reprogramming recommendations can improve operation of the underlying hardware.

Parameter settings can be tracked, including patterns of changes across different patients and resulting impact on cardiac capture. Capture can include confirmed capture of one or more chambers, such as confirmed LV cardiac capture, confirmed RV cardiac capture, confirmed RA cardiac capture, or combinations thereof (e.g., confirmed Bi-V cardiac capture, including RV and LV, confirmed LV-only, etc.). In an example, confirmed cardiac capture in less than 98% of cardiac beats (or in other examples, less than 95% or less than 90%) over a period of time, such as a week, a day, a group of successive beats, etc., can trigger an alert or notification and analysis or re-analysis of device parameter settings. In other examples, a reduced trend of confirmed cardiac capture over time, or a sudden loss of cardiac capture below a threshold (e.g., from above 98% to lower than 90%, 80%, 50%, 20%, 10%, or to 0%, etc.) can trigger an alert or notification and analysis or re-analysis of device parameter settings. In other examples, all sets of parameter settings can be analyzed with respect to model parameter settings to identify suboptimal programming and suggest changes, in certain examples, additionally with respect to confirmed cardiac capture percentage. For example, if key opinion leaders change a model set of parameter settings, even in situations where a patient has confirmed cardiac capture at 98% or above, a notification can be provided to a clinician illustrating the differences and impact of such to the medical device and the patient.

In addition, during programming or reprogramming of medical devices, related parameter settings can be identified and grouped, such as to suggest corresponding changes (e.g., during initial programming, during follow-up sessions, etc.), where one parameter setting is changed, suggesting corresponding changes to one or more other programmer settings to optimize therapy to the patient. In addition, values of such parameter settings can be analyzed to suggest not only specific parameter settings to program or reprogram, but values of different parameters based upon proposed changes. For example, if changing one parameter by a first amount, a recommendation can be provided to change a second parameter by a second amount. In contrast, if changing the one parameter by an amount greater than the first amount, the recommendation can be changed to change the second or one or more other parameters by an among different than the second amount, greater or less, depending on the specific parameters.

A recommendation system, such as comprising one or more assessment circuits, can utilize artificial intelligence and machine learning models to identify corresponding changes, for example, corresponding to positive outcomes (or away from negative outcomes) with respect to confirmed cardiac capture percentages of cardiac beats in other patients. In certain examples, parameter settings by key opinion leaders can be weighed more heavily than parameter settings by other clinicians. In other examples, specific models based on parameter settings of key opinion leaders, including individual clinicians or groups of clinicians at the forefront of thought leadership in the field of cardiac rhythm management therapy, can be suggested, separate from other clinicians. Combinations of parameter settings (e.g., including patterns, values, etc.) can be identified and validated to optimize cardiac capture, and validated identified parameter settings can be recommended as optimal values to clinicians, such as when programming or reprogramming medical devices, including implantable medical devices configured to provide cardiac resynchronization therapy (e.g., CRT devices), etc. In certain examples, validation can include combinations of human and machine validation confirmed based on stored information and recorded patient outcomes.

In other examples, other potential suboptimal pacing scenarios can be improved using the systems and methods described herein, such as identifying suboptimal parameter settings associated with high pacing (e.g., a percentage of paced beats above a high or desired pacing threshold) in an implantable cardioverter defibrillator (ICD) (e.g., not a cardiac rhythm management device, but a defibrillator), or chronic overpacing in one or more ambulatory medical devices, where a patient condition could have changed such that existing settings, although resulting in cardiac capture, are not required any longer. For example, patient physiologic information, such as heart sound information (e.g., S1 amplitude, providing an indication of cardiac contractility of a ventricle, etc.) can be used to triage patients and determine whether periods of intrinsic activity should be allowed, or if one or more parameter settings should be adjusted or recommended commensurate with a change in the patient physiologic information or patient status determined using such information.

FIG. 2 illustrates a table 201 of example parameter settings. In an example, specific parameter settings for a medical device can be represented by changes or deviation from a mode (e.g., a most frequent, or in other examples another statistical parameter, such as an average, a median, etc.) for a respective parameter setting from other medical devices, other medical devices in similar patients (e.g., grouped by disease, physiologic information, patient status, or combinations thereof), or the same medical device in a patient but at a different times. For example, whereas each medical device has a set of parameter settings, respective medical devices also have different sets of parameter settings at different times.

A kernel is a type of function used in various AI and machine learning algorithms that enables an algorithm to operate in high-dimension space without computing the coordinates of the data in that space. For example, a kernel can compute a dot product of multiple vectors without having to compute the coordinates of the points in that space, saving computational resources. The present inventors have recognized, among other things, that a kernel can be used to extract and store patterns in different sets of parameter settings of cardiac resynchronization therapy or other medical devices (e.g., an implantable cardioverter defibrillator (ICD), etc.) and to identify rules for different sets of parameters for specific sets or classes of devices performing similar functions, in certain examples, further tailored to specific patients based on physiologic information of such patients.

In FIG. 2, parameter settings for one medical device are illustrated in the table 201 across different office visits. The changes in settings are illustrated with a 1 or a 0 in the table 201. Columns or rows of the table 201, or in certain examples the table 201 itself, depending on organization, can be treated as vectors. In an example, columns A-D illustrate four different table entries. However, the four table entries A-D illustrate three parameter settings, P1-P3, with P1 having two states, reflecting different changes, for example, from a mode (most used value or setting for a particular parameter across a group of patients, such as in the training data, etc.) to a first value, illustrated as P1A, or from the mode to a second value, illustrated as P1B.

In a particular example, P1 can include an offset value, such as a sensed atrioventricular delay (AVD) offset value of 30, 40, or 50 ms, with a mode of 30 ms. PIA can represent a change from 30 ms to 40 ms, and P1B can represent a change from 30 ms to 50 ms. In other examples, additional table entries can be included, such as PIC illustrating a change from 40 ms to 50 ms, or other values, parameters, etc. P2 and P3 can include other parameter settings, such as particular sensing or tracking modes with 0 representing the mode (e.g., on or off) and 1 representing a change from the mode (e.g., off or on, respectively). As with P1 including PIA and P1B, P2 and P3 can also include other states as needed.

Parameter settings can be transformed into a table format or vectors, such as described herein (e.g., variance from a mode), and different combinations of settings and changes resulting therefrom (e.g., % of atrial or ventricular cardiac capture, etc.) can be evaluated to identify sets of parameter values or corresponding changes that represent the greatest positive impact, or positive impact above a threshold.

For example, FIG. 2 additionally illustrates a reduction 202 of combinations of parameter settings, that changes in parameter settings can be evaluated to identify candidate rules or parameters values or combinations of parameter values that exceed a minimum support value (a) above a threshold (e.g., an upper percentile of response, etc.). The minimum support value can be a function of frequency, impact, or combinations thereof.

In certain examples, upward closed analysis can be used to reduce the overall work required to identify combinations of parameter values and associated rules. Upward closed analysis generally refers to a property where, if first and second parameter values are below a minimum support value (e.g., P(A)<α and P(B)<α), then any combination of parameters including the first and second parameter values will be below the minimum support value (P(A,B)<α). Based on such analysis, the reduction 202 in FIG. 2 illustrates that if P(D)<α, then all combinations including P(D) can be excluded from analysis. Similarly, if P(B,C)<α, then all combinations including P(B,C) can be excluded from analysis.

Although described herein with respect to upward closed analysis, in other examples other types of analysis can similarly be used, such as lower closed analysis or other order or lattice theory elements or properties, etc.

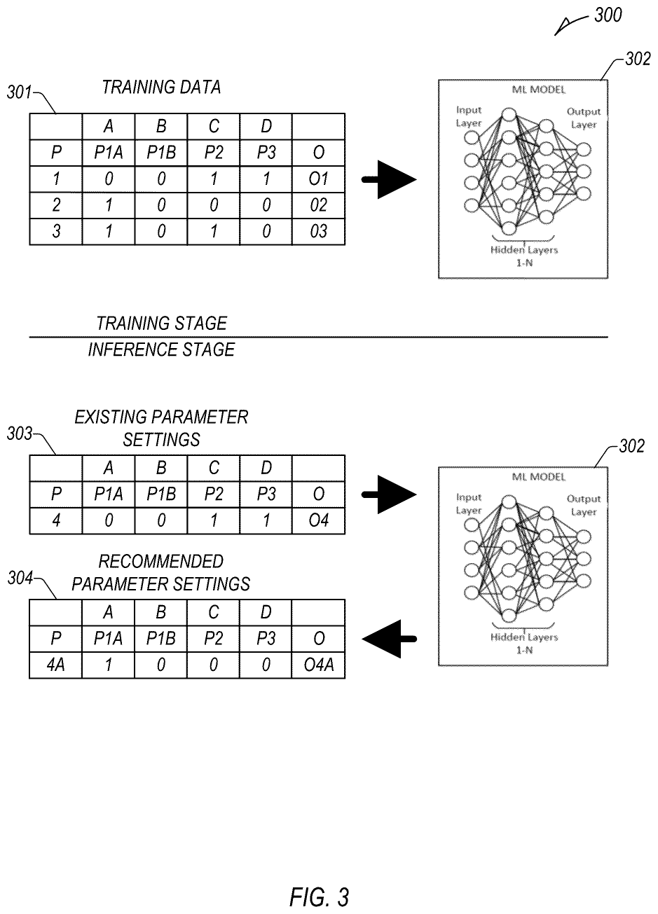

FIG. 3 illustrates a diagram of an example machine learning model 302 trained to receive, as input, training data 301, such as parameter settings from one or more ambulatory medical devices, and in certain examples physiologic information associated with respective parameter settings, such as associated cardiac capture rates of different patients, to identify optimal or suboptimal parameter settings or combinations of parameter settings corresponding to successful cardiac resynchronization therapy, and to generate an output indicating proposed parameter settings to optimize cardiac resynchronization therapy or to reduce suboptimal, missed, or reduced pacing based on existing parameter settings.

In the training (or learning) stage of FIG. 3, the training data 301 is applied to a machine learning algorithm to train the machine learning model 302. The training data 301 comprises parameter settings (e.g., P1A, P1B, P2, P3, etc., such as described in FIG. 2) from a number of different medical devices (e.g., 1-3, etc.). Although illustrated as a small number of parameters settings (e.g., P1A, P1B, P2, P3) and medical devices (1-3), in practice the training data 301 can include substantially more parameter settings and medical devices, such as tens or hundreds of parameter settings and hundreds or thousands of medical devices. In certain examples, the same medical device can provide parameter settings at different times. In certain examples, each vector or set of parameter settings can include an outcome (O) or assessment information, such as patient physiologic information (e.g., cardiac capture information, etc.) or other information of or about a patient associated with the respective parameter settings. In certain examples, the assessment can be provided by or validated by a user, such as based on direct measurement of patient physiologic information, etc.

The machine learning model 302 can be trained using supervised learning, unsupervised learning, or reinforcement learning. Examples of machine learning model architectures and algorithms may include, for example, decision trees, neural networks, support vector machines, or deep neural networks, etc. Examples of deep neural networks can include a convolutional neural network (CNN), a recurrent neural network (RNN), a deep belief network (DBN), a long-term and short-term memory (LSTM) network, a transfer learning network, or a hybrid neural network comprising two or more neural network models of different types or different model configurations. The training of the machine learning model may be performed continuously or periodically, or in near real time as additional patient data are made available. The training involves algorithmically adjusting one or more model parameters or parameter settings, until the model being trained satisfies a specified training convergence criterion. The trained machine learning model 302 can establish a correspondence between parameter settings or combinations of parameter settings and cardiac capture.

In some examples, a machine learning model can be trained to analyze physiological signal data and detect cardiac capture or successful or optimal cardiac resynchronization therapy, or correspondingly, suboptimal cardiac resynchronization therapy. To train such a model, a dataset is assembled containing sample patient physiologic information annotated by medical experts to identify successful cardiac capture and correspondingly, unconfirmed cardiac capture or unsuccessful cardiac capture. This annotated training dataset is then used to train the machine learning model 302 using a supervised learning approach, such as by algorithmically adjusting internal parameters to map the input patient physiologic information to the expert-applied labels. Various machine learning algorithms can be used, such as described above. The training process continues until the model achieves a high accuracy in classifying optimal or sub-optimal cardiac resynchronization therapy.

The training and utilization of machine learning models to determine indications of cardiac capture and to identify parameters or combinations of parameters associated with optimal or sub-optimal cardiac resynchronization therapy will depend on the specific type of ambulatory medical device being used and the nature of the physiological signal data it collects. Once trained, the machine learning model can receive new parameter settings and patient physiologic information as input and automatically detect if the patterns reflect optimal or sub-optimal cardiac resynchronization therapy and to identify specific parameter settings or combinations of parameter settings to change to optimize cardiac resynchronization therapy. This allows advanced cardiac resynchronization therapy analysis without needing to hard-code detection criteria. The model can be periodically retrained on new data to optimize cardiac resynchronization therapy over time.

In the inference stage of FIG. 3, for example, after the machine learning model 302 has been trained to reliably analyze parameter settings, patient physiologic information, or combinations thereof, and to identify and suggest programming or reprogramming recommendations to optimize cardiac resynchronization therapy in a training dataset and/or an evaluation dataset, the trained machine learning model 302 can be deployed for use in a production setting. Accordingly, existing parameter settings 303 (P1A, P1B, P2, P3) of an ambulatory medical device (4) can be provided as input to the trained machine learning model 302, such as at a remote server computer, in certain examples including patient physiologic information (O4). The trained machine learning model 302 can generate one or more outputs, including recommended parameter settings 304, indicating reprogramming recommendations for the ambulatory medical device (4A) and in certain examples an indication of outcome expected by such reprogramming recommendations (O4A). In certain examples, the received patient physiologic information can be used as input to a rule-based recommendation engine for generating a recommendation to reprogram an ambulatory medical device, for example, when one or more parameter settings or combinations of parameter settings are identified by the machine learning analysis as suboptimal or having a high likelihood of resulting in suboptimal cardiac resynchronization therapy.

Consistent with some embodiments, a reprogramming recommendation may involve a recommendation to modify or reprogram the device to use one or more different settings in sensing events, activity, or physiologic information, such as one or more blanking periods, thresholds, etc., or in providing stimulation, such as one or more intervals, delays, stimulation amplitudes, selected electrodes or vectors, etc. In certain examples, the recommendation to modify or reprogram the device can include a recommendation to use one or more different sensitivity settings or modes different from the current sensitivity setting or mode. With some embodiments, each sensitivity setting, or sensitivity mode is associated with one or more predefined threshold values. Accordingly, when a device is reprogrammed to operate in a new sensitivity mode, one or more of the predefined threshold values can change, thereby increasing or decreasing the sensitivity for detecting a particular event or activity. The reprogramming recommendation may be presented via a user interface of a software application to a clinician, who may undertake the task of reprogramming the device for a patient. The clinician can then evaluate the recommendation and reprogram the ambulatory medical device accordingly. This allows the clinician to validate any proposed changes to the device based on their expert judgment. In other examples, the reprogramming recommendation can be automatically applied within limits, such as previously validated by the clinician, etc.

Embodiments of the present invention provide numerous technical advantages for optimizing programming of and therapy delivery by ambulatory medical devices. By periodically evaluating collected physiological signal data using advanced machine learning models, embodiments of the invention enable closed-loop optimization of operation of the ambulatory medical device over time. This allows enhancing operation of the ambulatory medical device compared to relying solely on static detection settings programmed at implantation. The machine learning analysis can identify suboptimal parameter settings missed by the ambulatory medical device or the clinician programming the ambulatory medical device and recommend adjustments to improve operation when appropriate. The system can adapt parameter settings to the individual patient commensurate with patient status or therapy efficacy, providing more accurate therapy and without requiring constant manual reprogramming, reducing workload for clinicians while optimizing device performance and increasing the speed of training and sharing updated protocols and guidance.

Parameter settings differ based on the type of medical devices and in certain examples can include different modes of therapy. For example, different modes of cardiac resynchronization pacing include DDD and DDDR pacing, among others. The first “D” in DDD pacing represents dual (D) chamber (atrium and ventricle) pacing, the second “D” represents dual (D) chamber sensing, and the third “D” represents dual (D) chamber response to sensing in coordinating contraction of the heart and improve cardiac function. The additional “R” in DDDR pacing represents rate (R) modulation, where the medical device can adjust the pacing rate based on physiologic information indicating activity or need (e.g., activity, breathing rate, etc.).

Parameter settings can include, among others: Sensed Atrioventricular Delay Offset (SenAVDIyOffset or SAVDO), which adjusts the delay after a sensed atrial event; Atrioventricular Dynamic Minimum (AVDynMin or AVDM), which sets the minimum dynamic atrioventricular delay; Atrioventricular Delay Fixed (AVDlyFix or AVDF), which is a fixed atrioventricular delay setting; Atrioventricular Dynamic Maximum (AVDynMax or AVDM), which defines the maximum dynamic atrioventricular delay; Lower Rate Interval (LRLIntvl or LRLI), which sets the minimum pacing rate for the device; Left Ventricular Offset (LVOffset or LVO), which adjusts the timing of left ventricular pacing in relation to right ventricular pacing; Atrioventricular Dynamic Enable (AVDynEnbl or AVDE), which enables dynamic adjustment of the AVD; and Maximum Tracking Rate Interval (MTRIntvl or MTRI), the fastest interval at which the device will track atrial rates.

Additional parameter settings can include: Atrial Tachy Response Mode (ATRMode or ATRM), which defines the operational mode of the atrial channel; Biventricular Trigger Enable (BiVTrigEnbl or BVTE), which activates the biventricular pacing trigger; Ventricular Tachycardia Zone Rate (VTZoneRate or VTZR), which sets the rate threshold for detecting ventricular tachycardia; Atrial Tachy Response Trigger Rate (ATRTrigRt or ATRTR), which is the rate at which

Atrial Tachy Response Mode is triggered; Maximum Sensor Rate Interval (MSRIntvl or MSRI), the maximum rate at which the device will pace in response to sensor input; Ventricular Tachycardia 1 Zone Rate (VT1ZoneRate or VT1ZR), which specifies the rate for a particular zone of ventricular tachycardia detection; Number of Ventricular Zones (NumVZones or NVZ), which determines how many zones are used for ventricular tachyarrhythmia detection; Ventricular Fibrillation Zone Rate (VFZoneRate or VFZR), which sets the rate threshold for detecting ventricular fibrillation; Atrial Tachy Response Ventricular Rate Regulation Response (ATRVRRResp or ATRVRRR), a setting that adjusts the ventricular pacing rate in response to atrial rate; Atrial Tachy Response Biventricular Trigger Enable (ATRBiVTrigEnbl or ATRBVTE), which allows for biventricular pacing in response to atrial rate; Atrial Tachy Response Lower Rate Limit (ATRLRL), which sets the minimum pacing rate in ATR mode; Tachycardia Mode (TachyMode or TM), which defines the operational mode for tachycardia detection and therapy; Respiration Rate Trend Enable (RRTenable or RRT), which activates the respiration rate tracking feature; Atrial Tachy Response Pacing Chamber (ATRPaceCham or ATRPC), which specifies the chamber to be paced in atrial tachy mode; and Sensing Mode (SenseMode), which determines how the device senses cardiac events.

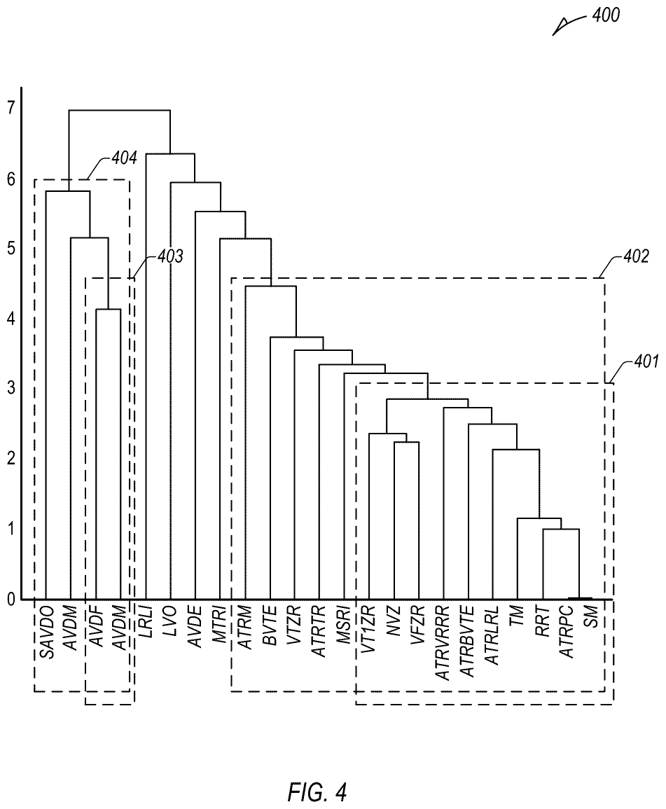

FIG. 4 illustrates a dendrogram 400 of identified hierarchical relationships between different parameter settings, such as those described above and presented on the horizontal axis of the dendrogram 400, with respect to cardiac capture. Each branch represents a cluster or combination of different parameter settings, and the length of the branches between the settings reflects the degree of similarity (or dissimilarity) between the clusters, with shorter lengths indicating a stronger relationship. Here, in the context of this application, a stronger relationship indicates a greater positive impact to moving the cluster or combination of parameters in the same programming session (e.g., if moving one, recommend also moving the other) with respect to optimized CRT.

For example, the dendrogram 400 in FIG. 4 illustrates four subgroups, a first subgroup 401, a second subgroup 402, a third subgroup 403, and a fourth subgroup 404. The first, second, and third subgroups 401, 402, 403 each have a degree of similarity less than 4.5. The first subgroup 401 includes the following close relationships, suggesting that such parameter settings should be grouped, for example, using one or more rules executed by the programmer: (1) Sensing Mode (SenseMode) and Atrial Tachy Response Pacing Chamber (ATRPaceCham or ATRPC) should be adjusted or recommended to be adjusted together, for example, on the same or sequential programming screens, followed closely by; (2) Respiration Rate Trend Enable (RRTenable or RRT); (3) Tachycardia Mode (TachyMode or TM), (4) Atrial Tachy Response Lower Rate Limit (ATRLRL).

The first subgroup 401 additionally illustrates strong relationships between (1)-(4) and the following: (5) Atrial Tachy Response Ventricular Rate Regulation Response (ATRVRRResp or ATRVRRR) and (6) Atrial Tachy Response Biventricular Trigger Enable (ATRBiVTrigEnbl or ATRBVTE). Somewhat unexpectedly, (7) Number of Ventricular Zones (NumVZones or NVZ); (8) Ventricular Fibrillation Zone Rate (VFZoneRate or VFZR); and (9) Ventricular Tachycardia 1 Zone Rate (VT1ZoneRate or VT1ZR) are more closely related to each other than (1)-(6). The second subgroup 402 includes the close relationships of the first subgroup 401 (1)-(9) and additionally the following: (10) Maximum Sensor Rate Interval (MSRIntvl or MSRI); (11) Atrial Tachy Response Trigger Rate (ATRTrigRt or ATRTR); (12) Ventricular Tachycardia Zone Rate (VTZoneRate or VTZR); (13) Biventricular Trigger Enable (BiVTrigEnbl or BVTE); and (14) Atrial Tachy Response Mode (ATRMode or ATRM).

The third subgroup 403 illustrates a strong relationship between Atrioventricular Delay Fixed (AVDlyFix or AVDF) and Atrioventricular Dynamic Maximum (AVDynMax or AVDM), suggesting that such parameter settings should be grouped using one or more rules. The fourth subgroup 404, although having a value less than 6 in contrast to a value less than 4.5 for the second and third subgroups 402, 403 and a value of less than 3 for the first subgroup 401, somewhat unexpectedly groups adds Sensed Atrioventricular Delay Offset (SenAVDIyOffset or SAVDO) and Atrioventricular Dynamic Minimum (AVDynMin or AVDM) to the relationships of the third group 403. Each of the identified subgroups (e.g., the first subgroup 401, the second subgroup 402, the third subgroup 403, and the fourth subgroup 404) have significant identified relationships and can be changed or alerted to be changed together, such that if one setting from the group is adjusted, the others are alerted for consideration or adjustment.

As described herein, patterns or combinations of parameter settings can be identified and validated as affecting or impacting cardiac resynchronization therapy. For example, groups of parameters can be identified having a higher percentage of cardiac capture rates than other parameters, or groups that, once such parameter settings are implemented, show an increase in rates of cardiac capture. Once identified, individual values for specific parameter settings can be toggled or changed and the impact to cardiac resynchronization therapy across a population or number of patients can be analyzed to determine positive or negative impact on delivered therapy, such as evidenced by rates of cardiac capture or other patient physiologic information.

In one example, a first group of parameter settings was identified as: VTZoneRate=300 ms, SenAVDlyOffset=40 ms, NumVZones=3. Once implemented, an increase in LV cardiac capture was detected. To validate the identified group, cardiac capture rates (e.g., LV pacing % (successful capture) and RV pacing %) are analyzed by toggling values of the parameter settings to or away from default or mode values for each setting and comparing cardiac capture rates for different values. If the cardiac capture rates do not significantly change (e.g., increase or decrease less than a threshold percentage) with respect to the toggle, the specific parameter value can be identified as not having a substantial impact. However, if the cardiac capture rates change (e.g., an increase or decrease more than a threshold percentage) with respect to the toggle, the specific parameter value can be identified as having a substantial impact. In this example, the VTZoneRate=300 ms parameter (e.g., toggled with respect to an example mode value of 400 ms, etc.) provided a more substantial positive impact on LV cardiac capture rate than the other parameters, though less than the combination of the first group of parameter settings in aggregate, validating the combination.

In contrast, in another example, a second group of parameter settings was identified as: BiVTrigEnbl=1, SenAVDlyOffset=50 ms, VFZoneRate=273 ms, NumVZones=3. Once implemented, an increase in RV cardiac capture was detected. To validate the identified group, cardiac capture rates are analyzed by toggling values of the parameter settings to or away from default or mode values for each setting and comparing cardiac capture rates for different values. In this example, the BiVTrigEnbl=1 parameter (e.g., toggled from 1 to 0) provided a substantial positive impact on LV cardiac capture and the NumVZones=3 parameter (e.g., toggled from 3 to 2, etc.) provided a substantial positive impact on RV cardiac capture, though each less than the combination of the second group of parameter settings in aggregate, validating the combination.

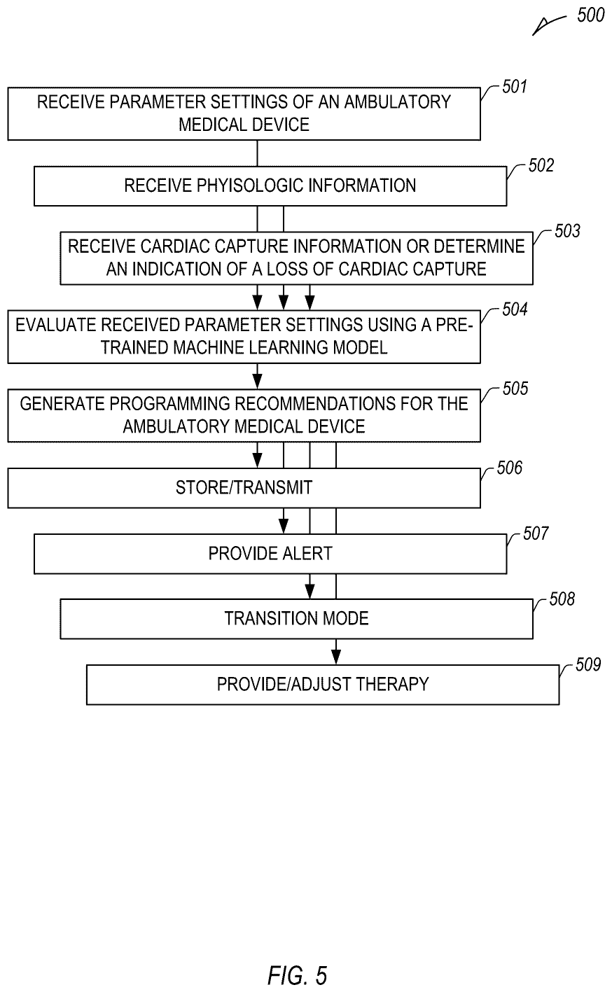

FIG. 5 illustrates an example method 500 of programming, reprogramming, or generating a reprogramming recommendation for an ambulatory medical device to improve cardiac capture in a patient during cardiac resynchronization therapy by the ambulatory medical device.

At step 501, parameter settings of an ambulatory medical device (e.g., an implantable cardiac resynchronization therapy device) are received, such as using a signal receiver circuit of a patient management system. The parameter settings can include proposed parameter settings input by a clinician during a programming or reprogramming session, existing parameter settings of an operational ambulatory medical device, or other parameter settings of or proposed for an ambulatory medical device.

At step 502, physiologic information of a patient can be received, such as using a signal receiver circuit of a sensor, an implantable medical device, an ambulatory medical device, or a component of the patient management system. The physiologic information can include one or more types of physiologic information sensed using one or sensors of an implantable medical device, such as described herein. In one example, the received physiologic information can include respiration information sensed using one or both of an accelerometer or an impedance sensor. In other examples, the received physiologic information can include other information, such as heart sound information, activity information, heart rate information, etc., sensed using one or more sensors of an implantable medical device.

At step 503, cardiac capture information can be received, such as using the signal receiver circuit, or an indication of a loss of cardiac capture of a heart of the patient can be determined, such as using an assessment circuit of the patient management system. In an example, the received physiologic information can be used to determine an indication of cardiac capture of the heart of the patient, or correspondingly to determine the loss of cardiac capture of the heart of the patient, in response to applied cardiac resynchronization therapy pacing, etc.

At step 504, the received parameter settings can be evaluated using one or more pre-trained machine learning models, such as using one or more assessment circuits of the patient management system, including, for example, one or more remote devices, etc. The one or more pre-trained machine learning models can be trained, in certain examples, such as described herein, for example, with respect to the process illustrated and described in FIG. 3.

For example, the received parameter settings be processed by inputting the received parameter settings into one or more pre-trained machine learning models, each of the one or more pre-trained machine learning models trained to compare the received parameter settings to stored model parameter settings from one or more other ambulatory medical devices corresponding to one or more other patients and to identify one or more differences between the parameter settings of the ambulatory medical device and the stored model parameter settings of the one or more other ambulatory medical devices. Upon obtaining an output from the one or more pre-trained machine learning models indicating the identified one or more differences between the parameter settings of the ambulatory medical device and parameter settings of the one or more other ambulatory medical devices, a programming recommendation can be generated for the ambulatory medical device to improve cardiac capture for the patient based on the identified one or more differences.

At step 505, one or more programming recommendations can be generated for the ambulatory medical device, such as by applying the one or more pre-trained machine learning models to generate proposed changes to existing parameter settings or additional parameter settings to change, including values and settings particular to the ambulatory medical device, the patient, or with respect to the received parameter settings. Rules can be determined such that, if a first change or parameter setting is proposed, a second change or parameter setting is proposed or suggested to be changed commensurate with the first change. In certain examples, the rule can include providing data illustrating the potential detriment of the received parameter settings or a proposed benefit of additional recommendations. In certain examples, a rule can include a warning or an alert provided to the clinician with the potential detriment, benefit, or recommendation. In other examples, if no additional programming recommendation or changes are determined, such that the one or more pre-trained machine learning models are in agreement, a confirmation can be provided to the clinician, indicating agreement with the received parameter settings. As changes to the parameter settings are made during this process, updated parameter settings can be received and evaluated to determine agreement or additional recommendations.

In certain examples, the programming recommendations can be generated and provided at the programmer itself, during programming, such as by alerts or notifications during programming. In certain examples, groups of parameter settings shown to beneficially move together can be grouped into the same or sequential programmer screens on the user interface used by clinicians when programming the ambulatory medical device.

At step 506, the generated programming (or reprogramming) recommendations can be provided, such as by the assessment circuit, through one or more communication circuits, etc., to a user or process, such as providing an output of programming (or reprogramming) recommendations to a user interface for display to the user or to a control circuit to control or adjust the process or function of the medical device system, etc. The recommendations can be stored, such as using the assessment circuit, and transmitted, by control of the assessment circuit or using one or more communication circuits, etc., such as to one or more additional processes or components, such as an output circuit (e.g., a display, a controller for a display, etc.).

In an example, when parameter settings are analyzed using one or more trained machine learning models, and one or more suboptimal parameter settings or combinations of parameter settings are identified, a recommendation to reprogram the medical device may be generated and presented to a clinician via a user interface of the remote device, or via a user interface of a software application executing on a client device communicatively connected with the remote device, in certain examples including proposed parameter settings to optimize cardiac resynchronization therapy or to reduce suboptimal, missed, or reduced pacing.

At step 507, an alert can be optionally provided, such as by the assessment circuit, for example, if the programming (or reprogramming) recommendations are available for review or transmission, if one or more changes in patient physiologic information or determined outcomes are determined or detected, such as above a threshold, etc. In an example, an output can be provided of the programming (or reprogramming) recommendations to a user interface for display to a user or to another circuit to control or adjust a process or a function of an implantable or ambulatory medical device, such as to adjust a follow-up schedule associated with the patient, a clinician, etc.

At step 508, one or more modes or functions of the assessment circuit or an implantable or ambulatory medical device can be optionally adjusted based on one or more of the programming (or reprogramming) recommendations or patient physiologic information, etc. In other examples, after a set amount of time, or once one or more measures of physiologic information indicates that cardiac resynchronization therapy is stable or changing, etc., one or more modes or functions of the implantable or ambulatory medical device can be altered to increase or decrease a power consumption or sensing or storage capability of the implantable or ambulatory medical. For example, one or more hardware limitations can be adjusted, such as to, among others: sense or receive more or less physiologic information of the patient; increase communication frequency between the implantable or ambulatory medical device and an external device (e.g., remote device, programmer, etc.), such as to increase the frequency of patient monitoring, etc.; switch to a different or more power or resource intensive monitoring algorithm; etc.

At step 509, one or more therapies can be optionally provided or adjusted based on the determined programming (or reprogramming) recommendations or one or more other measures, values, parameter settings, or metrics, such as described herein.

Although illustrated as a method from step 501 through step 509, in certain examples, one or more steps are options, and in other examples, different combinations or permutations of these or other steps or examples can be combined to form other methods or processes, which is also applicable to other examples discussed herein.

### Operation Modes

Ambulatory medical devices powered by rechargeable or non-rechargeable batteries, responsible for sensing physiologic signals and physiologic information of the patient, and in certain examples making determinations using such information, have to make certain tradeoffs between device battery life, or in the instance of implantable medical devices with non-rechargeable batteries, between device replacement periods often including surgical procedures, and device sensing, storage, processing, and communication characteristics, such as sensing resolution, sampling frequency, sampling periods, the number of active sensors, the amount of stored information, processing characteristics, or communication of physiologic information outside of the device.

Medical devices can include higher-power modes and lower-power modes. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode.

A technological problem in the art with respect to such devices exists that not all information can be stored, not all sensors can be active in a high-power or high-resolution mode, not all algorithms can be active, and not all sensed or processed information can be communicated outside of the device at all times without detrimentally impacting the lifespan of the devices. Technological solutions to such problems are often improvements in physical sensors, or alternatively in sensing and processing physiologic information in a way that improves device efficiency, extending the lifespan of the device, or to perform new determinations using existing sensors or information in a way that was not previously known, increasing the capabilities of an existing device without adding additional hardware to the device, or requiring additional sensors or hardware to be implanted in the patient. Efficiency improvements in one area can enable additional operation in another, improving the technical capabilities of existing devices having real-world constraints.

For example, physiologic information, such as indicative of a potential adverse physiologic event, can be used to transition from a low-power mode to a high-power mode. However, by the time physiologic information detected in the low-power mode indicates a possible event, valuable information has been lost, unable to be recorded in the high-power mode.

Another technological problem exists in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

In an example, a change in modes can enable higher resolution sampling or an increase in the sampling frequency or number or types of sensors used to sense physiologic information leading up to and including a potential event. Different physiologic information is often sensed using non-overlapping time periods of the same sensor, in certain examples, at different sampling frequencies and power costs.

For example, ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiration rate, phase, breathing sounds, etc.), etc. In one example, heart sounds and patient activity can be detected using non-overlapping time periods of the same, single- or multi-axis accelerometer, at different sampling frequencies and power costs.

In an example, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

Another technological problem exists in that suboptimal programming of device parameters and parameter settings can negatively impact functionality of ambulatory medical devices. Accordingly, identifying suboptimal programming by clinicians and other caregivers and generating and providing alerts or notifications of such identified suboptimal programming, or reprogramming recommendations, and in certain examples, reprogramming ambulatory medical devices directly, can improve the functionality of existing ambulatory medical devices without requiring other improvements to the hardware of devices providing therapy or the sensors themselves.

### Physiologic Information

Heart sounds are recurring mechanical signals associated with cardiac vibrations or accelerations from blood flow through the heart or other cardiac movements with each cardiac cycle and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a measure (e.g., a ratio) of respiratory frequency relative to (e.g., divided by) tidal volume of the patient. The nHR is a measure of heart rate (HR) of the patient at night, either in relation to sensing patient sleep or using a preset or selectable time of day corresponding to patient sleep. In certain examples, respiration information of the patient can be determined using changes in impedance information and accordingly can be considered electrical information, but different than cardiac electrical information. In other examples, respiration information of the patient can be determined using changes in activity or acceleration information and accordingly can be considered mechanical information.

Physiologic metrics, as described herein, or measures or indications of physiologic information, can include one or more different measures of rate, amplitude, energy, etc., of different physiologic information over one or more time periods, such as representative daily values, etc. For example, heart sound metrics can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily metrics can be determined representative of an average daily value for the patient, either corresponding to a waking time or a 24-hour period, etc. Respiration metrics can include, among other things, a mean or median respiration rate, binned values of rates, and a representative value of specific rate bins, etc. Heart rate metrics can include an average nighttime heart rate, a minimum nighttime heart rate, heart rate at rest, etc.

The activity information can include an activity measurement of the patient, such as detected using an accelerometer, a posture sensor, a step counter, or one or more other activity sensors associated with an ambulatory medical device. Activity may be used to gate other physiologic measurements such as heart rate or respiration rate so that the change in these metrics with increased patient activity may be used to infer patient cardiovascular and metabolic status including measurement of oxygen consumption. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In contrast to and separate from the electrical or mechanical information discussed above, the chemical information can include information about one or more chemical properties of blood, interstitial space (e.g., the space between cells, such as including interstitial fluid), or other tissue (e.g., muscle tissue, fat tissue, organ tissue, etc.) of the patient, such as information indicative of or including one or more of a glucose level, pH level, dissolved gas level (e.g. oxygen, carbon dioxide, carbon monoxide, etc.), electrolyte level (e.g., sodium, potassium, calcium, etc.), organic compound level (e.g., lactate, cholesterol, hemoglobin, creatinine, etc.), or biologic compound level (e.g., enzymes, antibodies, receptors, etc.), etc. The chemical information may be measured by one or more of an electrical sensor, mechanical sensor, electrochemical sensor, biosensor (e.g., enzyme biosensor, etc.), ion-selective electrode sensor, optical sensor, etc. In an example, the chemical information may include potassium information (e.g., one or more of interstitial potassium information, serum potassium information, etc.), creatinine information (e.g., one or more of interstitial creatinine information, serum creatinine information, etc.), or combinations thereof.

In certain examples, interstitial chemical information, such as one or more chemical levels in an interstitial space (e.g., a space between one or more of connective tissue, muscle fibers, nervous tissue, etc.) or of interstitial fluid, etc., can be indicative of serum chemical information. For example, potassium may move between cells or tissue and interstitial fluid (e.g., a change in interstitial potassium level may be followed by or reflective of a change in serum potassium level or vice versa), such that chemical information on serum potassium can include interstitial potassium. In certain examples, one of interstitial or serum chemical information can lead or lag the other, such that a change in one can indicate a worsening patient condition is detectable before the other. In one example, interstitial potassium information can lead serum potassium information as an indicator of electrolyte imbalance.

In certain examples, an alert state (e.g., an in-alert state, an out-of-alert state, a priority alert state, etc.) of the patient can be adjusted or determined using chemical information of the patient, such as to increase a sensitivity or specificity of alert state determination, reduce false positive alert state determinations, alert state transitions or adjustments, or otherwise reduce storage or transmission of physiologic information associated or transitions associated with false positive alert state determinations, and power and processing resources associated with the same. In an example, the alert state can be determined using a comparison of a value of the health index (e.g., a numerical value, etc.) to one or more fixed or adaptable alert thresholds (e.g., based at least in part on one or more relative factors, such as measurements from the patient over the past 30 days, etc.). In an example, the alert state can be provided to a user interface for display to a user or to a control circuit to control or adjust a process or function of the system. In an example, the alert state can include one or more of an indication, recommendation, or instruction to perform one or more actions (e.g., administer or provide a drug or class of drug, adjust or optimize a guideline-directed medical therapy (GDMT), etc.). For, example, a GDMT may advise administration of a quantity of a drug or a rate of increase in a dosage, etc. In an example, determination of an in-alert or priority alert state can trigger an indication or instruction to administer or provide a specific class of diuretic or to deviate from GDMT (e.g., increase GDMT above a standard recommendation, hold GDMT at a standard recommendation, hold GDMT at a current level, decrease GDMT below a standard recommendation, increase a dosage or rate of increase of a drug, reduce a dosage or rate of decrease of a drug, etc.).

In certain examples, the techniques described above or herein can be used in various combinations or permutations. For example, combinations or permutations of techniques described above or herein can be selected based upon patient history, patient treatment (e.g., in-patient care, out-patient care, etc.), clinician input, etc.

As used herein, high and low (or high, medium, and low, etc.) can be relative or categorical terms, in certain examples with respect to clinical or population values, patient-specific values (e.g., a representative value, such as a current value, with respect to a short- or long-term range of values, etc.), or combinations thereof. For example, a high value can include a value in an upper percentage (e.g., at or above an upper quartile, etc.) of values experienced by the patient over respective time periods, such as one or more of a short-term range (e.g., having a period between 1 week and 3 months, such as 1 month, etc.), a long term range (e.g., having a period greater than the short-term range, such as greater than 1 month, greater than 3 months, the last 6 months, or longer, etc.). A low value can include a value in a lower percentage (e.g., at or below a mean or median, below the upper quartile, etc.). A medium value can, in certain examples, include a value between the upper and lower quartiles or within a threshold percentage of a mean or median, etc. In other examples, values can be determined with respect to clinical or population values, in certain examples, further respective to matching patient demographics (e.g., age, sex, comorbidities, etc.) or type of medical device (e.g., CRT-D device, ICD device, etc.), etc.

In an example, determinations described herein can be used to change device behavior, trigger additional sensing, data processing, storage, or transmission, or otherwise alter one or more modes, processes, or functions of medical devices associated with such determinations. For example, determinations can require data over a substantial time period (e.g., multiple days, weeks, a month or more, etc.). Such determinations can be initially determined by the device at yearly or semi-yearly (e.g., every 6 months, every 3 months, etc.) by default, or triggered by worsening patient status or upon instruction from a clinician or caregiver, etc. In a first example, an assessment circuit can determine one or more indications quarterly, consuming a default amount of device resources. If the quarterly determination exceeds one or more of a patient-specific or population threshold, the assessment circuit can alter device functionality to increase the frequency of making such determinations, increasing the use of device resources, in certain examples reducing device lifespan, but providing additional monitoring and determinations. In other examples, if a determination exceeds one or more thresholds, additional sensing can be triggered, such as enabling additional sensors, or sensing enabled sensors with a higher resolution or sampling frequency, storing more information, and communicating more information outside of the device, such as to an external programmer, or increasing the frequency of communication outside of the device, increasing the use of device resources, in certain examples reducing device lifespan, but providing additional monitoring and determinations.

In certain examples, determinations described herein can include one or more determined risk curves illustrating determined risks at different time periods into the future, such as a determined risk of mortality (e.g., cardiovascular death), a determined risk of heart failure hospitalization, etc. Information about the determined risks or the determined risk curves or portions of the determined risk curves themselves can be provided to a user, such as to a patient, clinician, caregiver, etc., or can be used to make one or more device changes, such as described herein (e.g., therapies, treatments, device settings, etc.), or trigger one or more other processes or notifications, etc.

### Composite Indications of Patient Condition

Indications of patient condition can include single-feature determinations based on a single feature or measure of a single type of physiologic information, or separately a composite determination based on a combination of physiologic information, such as two or more separate features of physiologic measures. In addition, indications of patient condition can be device-based, such as determined using physiologic information detected from the patient using the one or more ambulatory medical devices without input of clinical information about the patient separate from that detected or sensed physiologic information. In other examples, indications of patient condition can be a combination of device-based and clinical-based information of the patient, such as clinician diagnosis or determination of risk, patient history, patient age, comorbidities, prior hospitalization, type of implanted device, etc. In certain examples, separate determinations can be made for different combinations of clinical information.

One example of a composite indication is a HeartLogic™ index, a HeartLogic™ in-alert time, or one or more other composite measurements or measures thereof. The HeartLogic™ index is a composite indication of patient condition determined using different combinations or weightings of physiologic information, including two or more of S1 heart sounds, S3 heart sounds, thoracic impedance, activity information, respiration information, and nighttime heart rate (nHR). The HeartLogic™ index can be indicative of a heart failure status, a risk a heart failure event (e.g., within in a given time period), or a worsening of the heart failure status or risk of heart failure event in the patient over time. The HeartLogic™ in-alert time is a measure of time that the HeartLogic™ index is above an alert threshold.

In certain examples, the different combinations or weightings of physiologic information used to determine the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.). For example, if the risk stratifier is low, or below a first threshold, the HeartLogic™ index can be determined using a first combination of physiologic information. If the risk stratifier is high, or above a second threshold, the HeartLogic™ index can be determined using a second combination of physiologic information, such as additional information than included in the first combination (e.g., the first combination and the second combination, etc.). If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination than the first combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

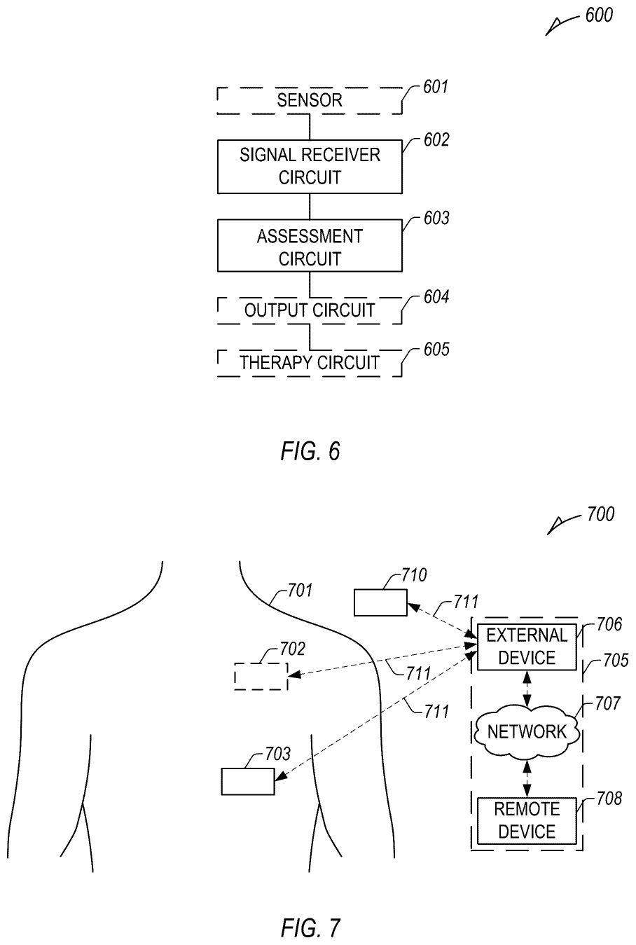

FIG. 6 illustrates an example system 600 (e.g., a medical device system). In an example, one or more aspects of the system 600 can be a component of, or communicatively coupled to, a medical device, such as an implantable medical device (IMD), an insertable cardiac monitor (ICM), an ambulatory medical device (AMD), etc. The system 600 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 600 can include a single medical device or a plurality of medical devices implanted in a body of a patient or otherwise positioned on or about the patient to monitor patient physiologic information of the patient using information from one or more sensors, such as a sensor 601. In an example, the sensor 601 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, a potassium sensor, a creatinine sensor, etc.); a temperature sensor; a skin elasticity sensor, or one or more other sensors configured to receive physiologic information of the patient.

The example system 600 can include a signal receiver circuit 602 and an assessment circuit 603. The signal receiver circuit 602 can be configured to receive physiologic information of a patient (or group of patients) from the sensor 601. The assessment circuit 603 can be configured to receive information from the signal receiver circuit 602, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information. In an example, the signal receiver circuit 602 can include the sensor 601. In other examples, the signal receiver circuit can be coupled to or a component of the assessment circuit 603.

In certain examples, the assessment circuit 603 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

In certain examples, the assessment circuit 603 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 600 can include an output circuit 604 configured to provide an output to a user, or to cause an output to be provided to a user, such as through an output, a display, or one or more other user interface, the output including a score, a trend, an alert, or other indication. In other examples, the output circuit 604 can be configured to provide an output to another circuit, machine, or process, such as a therapy circuit 605 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 605 can include one or more of a stimulation control circuit, a cardiac stimulation circuit, a neural stimulation circuit, a dosage determination or control circuit, etc. In other examples, the therapy circuit 605 can be controlled by the assessment circuit 603, or one or more other circuits, etc. In certain examples, the assessment circuit 603 can include the output circuit 604 or can be configured to determine the output to be provided by the output circuit 604, while the output circuit 604 can provide the signals that cause the user interface to provide the output to the user based on the output determined by the assessment circuit 603.

FIG. 7 illustrates an example patient management system 700 and portions of an environment in which the patient management system 700 may operate. The patient management system 700 can perform a range of activities, including remote patient monitoring and diagnosis of a disease condition, programming of ambulatory medical devices, and control of one or more therapies. Such activities can be performed proximal to a patient 701, such as in a patient home or office, through a centralized server, such as in a hospital, clinic, or physician office, or through a remote workstation, such as a secure wireless mobile computing device.

The patient management system 700 can include one or more medical devices, an external system 705, and a communication link 711 providing for communication between the one or more ambulatory medical devices and the external system 705. The one or more medical devices can include an ambulatory medical device (AMD), such as an implantable medical device (IMD) 702, a wearable medical device 703, or one or more other implantable, leadless, subcutaneous, external, wearable, or medical devices configured to monitor, sense, or detect information from, determine physiologic information about, or provide one or more therapies to treat various conditions of the patient 701, such as one or more cardiac or non-cardiac conditions (e.g., dehydration, sleep disordered breathing, etc.).

In an example, the implantable medical device 702 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 701. In another example, the implantable medical device 702 can include a monitor implanted, for example, subcutaneously in the chest of patient 701, the implantable medical device 702 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemakers can communicate between themselves, or one or more other implanted or external devices.

The implantable medical device 702 can include a signal receiver circuit or an assessment circuit configured to detect or determine specific physiologic information of the patient 701, or to determine one or more conditions or provide information or an alert to a user, such as the patient 701 (e.g., a patient), a clinician, or one or more other caregivers or processes, such as described herein. The implantable medical device 702 can alternatively or additionally be configured as a therapeutic device configured to treat one or more medical conditions of the patient 701. The therapy can be delivered to the patient 701 via the lead system and associated electrodes or using one or more other delivery mechanisms. The therapy can include delivery of one or more drugs to the patient 701, such as using the implantable medical device 702 or one or more of the other ambulatory medical devices, etc. In some examples, therapy can include CRT for rectifying dyssynchrony and improving cardiac function in heart failure patients. In other examples, the implantable medical device 702 can include a drug delivery system, such as a drug infusion pump to deliver drugs to the patient for managing arrhythmias or complications from arrhythmias, hypertension, hypotension, or one or more other physiologic conditions. In other examples, the implantable medical device 702 can include one or more electrodes configured to stimulate the nervous system of the patient or to provide stimulation to the muscles of the patient airway, etc.

The wearable medical device 703 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 705 can include a dedicated hardware/software system, such as a programmer, a remote server-based patient management system, or alternatively a system defined predominantly by software running on a standard personal computer. The external system 705 can manage the patient 701 through the implantable medical device 702 or one or more other ambulatory medical devices connected to the external system 705 via a communication link 711. In other examples, the implantable medical device 702 can be connected to the wearable medical device 703, or the wearable medical device 703 can be connected to the external system 705, via the communication link 711. This can include, for example, programming the implantable medical device 702 to perform one or more of acquiring physiologic data, performing at least one self-diagnostic test (such as for a device operational status), analyzing the physiologic data, or optionally delivering or adjusting a therapy for the patient 701. Additionally, the external system 705 can send information to, or receive information from, the implantable medical device 702 or the wearable medical device 703 via the communication link 711. Examples of the information can include real-time or stored physiologic data from the patient 701, diagnostic data, such as detection of patient hydration status, hospitalizations, responses to therapies delivered to the patient 701, or device operational status of the implantable medical device 702 or the wearable medical device 703 (e.g., battery status, lead impedance, etc.). The communication link 711 can be an inductive telemetry link, a capacitive telemetry link, or a radio frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 705 can include an external device 706 in proximity of the one or more ambulatory medical devices, and a remote device 708 in a location relatively distant from the one or more ambulatory medical devices, in communication with the external device 706 via a communication network 707. Examples of the external device 706 can include a medical device programmer. The remote device 708 can be configured to evaluate collected patient or patient information and provide alert notifications, among other possible functions. In an example, the remote device 708 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 708 can receive data from multiple patients. The data can be collected by the one or more ambulatory medical devices, among other data acquisition sensors or devices associated with the patient 701. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 708 may additionally include one or more locally configured clients or remote clients securely connected over the communication network 707 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 708, including the server and the interconnected clients, may also execute a follow-up scheme by sending follow-up requests to the one or more ambulatory medical devices, or by sending a message or other communication to the patient 701 (e.g., the patient), clinician or authorized third party as a compliance notification.

The communication network 707 can provide wired or wireless interconnectivity. In an example, the communication network 707 can be based on the Transmission Control Protocol/Internet Protocol (TCP/IP) network communication specification, although other types or combinations of networking implementations are possible. Similarly, other network topologies and arrangements are possible.

One or more of the external device 706 or the remote device 708 can output the detected medical events to a system user, such as the patient or a clinician, or to a process including, for example, an instance of a computer program executable in a microprocessor. In an example, the process can include an automated generation of a programming recommendation for an ambulatory medical device to improve cardiac capture for the patient. In an example, the external device 706 or the remote device 708 can include a respective display unit for displaying the physiologic or functional signals, or alerts, alarms, emergency calls, or other forms of warnings to signal the detection of one or more conditions. In some examples, the external system 705 can include a signal receiver circuit and an assessment circuit, such as an external data processor configured to analyze the physiologic or functional signals received by the one or more ambulatory medical devices, and to confirm or reject one or more determinations made by one or more ambulatory medical devices, such as the implantable medical device 702, the wearable medical device 703, etc., or make additional determinations, etc. Computationally intensive algorithms, such as machine-learning algorithms, can be implemented in the external data processor.

With some examples, when parameter settings of an ambulatory medical device are analyzed using one or more trained machine learning models, and one or more differences between the parameter settings of the ambulatory medical device and the stored model parameter settings are detected, a recommendation to reprogram the medical device may be generated and presented to a clinician via a user interface of the remote device 708, or via a user interface of a software application executing on a client device communicatively connected with the remote device 708. The recommendation to reprogram the medical device may be determined by identifying differences between the parameter settings of the ambulatory medical device and the stored model parameter settings via the one or more machine learning models that otherwise went undetected by a clinician or a medical device programmer.

Portions of the one or more ambulatory medical devices or the external system 705 can be implemented using hardware, software, firmware, or combinations thereof. Portions of the one or more ambulatory medical devices or the external system 705 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

A therapy device 710 can be configured to send information to or receive information from one or more of the ambulatory medical devices or the external system 705 using the communication link 711. In an example, the one or more ambulatory medical devices, the external device 706, or the remote device 708 can be configured to control one or more parameters of the therapy device 710. The external system 705 can allow for programming the one or more ambulatory medical devices and can receive information about one or more signals acquired by the one or more ambulatory medical devices, such as can be received via a communication link 711. The external system 705 can include a local external implantable medical device programmer. The external system 705 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected events can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), in certain examples, in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, for example, to increase arterial pressure, to maintain cardiac output, to disrupt or reduce the impact of the detected event, or combinations thereof.

In certain examples, physiologic information of a patient can be sensed using one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected event (e.g., a heart failure event, an arrhythmia event, etc.) can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

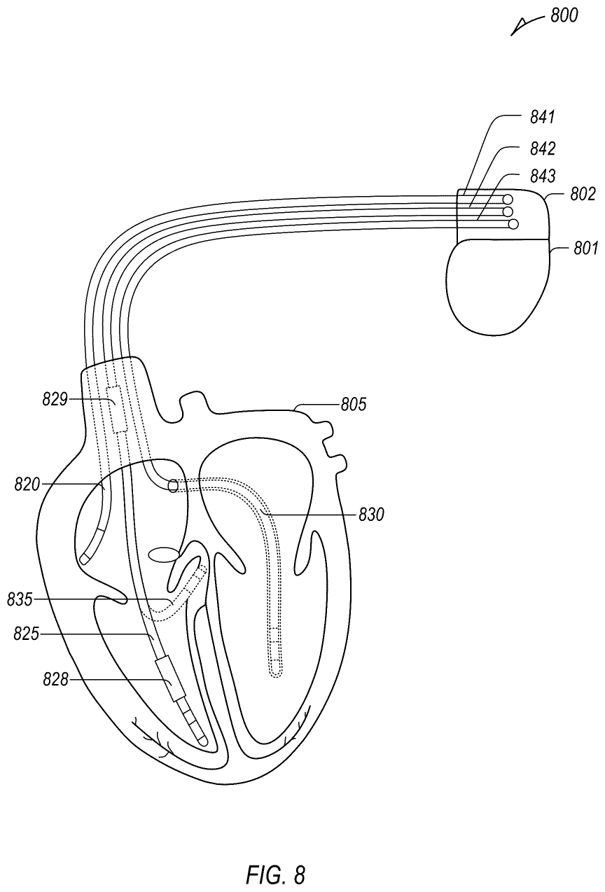

FIG. 8 illustrates an example implantable medical device (IMD) 800 electrically coupled to a heart 805, such as through one or more leads coupled to the implantable medical device 800 through one or more lead ports, including first, second, or third lead ports 841, 842, 843 in a header 802 of the implantable medical device 800. In an example, the implantable medical device 800 can include an antenna, such as in the header 802, configured to enable communication with an external system and one or more electronic circuits (e.g., an assessment circuit, etc.) in a hermetically sealed housing (CAN) 801.

The implantable medical device 800 may include an implantable cardiac monitor (ICM), pacemaker, defibrillator, cardiac resynchronization therapy (CRT) device, or other subcutaneous implantable medical device or cardiac rhythm management (CRM) device configured to be implanted in a chest of a subject, having one or more leads to position one or more electrodes or other sensors at various locations in or near the heart 805, such as in one or more of the atria or ventricles. Separate from, or in addition to, the one or more electrodes or other sensors of the leads, the implantable medical device 800 can include one or more electrodes or other sensors (e.g., a pressure sensor, an accelerometer, a gyroscope, a microphone, etc.) powered by a power source in the implantable medical device 800. The one or more electrodes or other sensors of the leads, the implantable medical device 800, or a combination thereof, can be configured detect physiologic information from, or provide one or more therapies or stimulation to, the patient.

The implantable medical device 800 can include one or more electronic circuits configured to sense one or more physiologic signals, such as an electrogram or a signal representing mechanical function of the heart 805. In certain examples, the CAN 801 may function as an electrode such as for sensing or pulse delivery. For example, an electrode from one or more of the leads may be used together with the CAN 801 such as for unipolar sensing of an electrogram or for delivering one or more pacing pulses. A defibrillation electrode (e.g., the first defibrillation coil electrode 828, the second defibrillation coil electrode 829, etc.) may be used together with the CAN 801 to deliver one or more cardioversion/defibrillation pulses.

In an example, the implantable medical device 800 can sense impedance such as between electrodes located on one or more of the leads or the CAN 801. The implantable medical device 800 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the implantable medical device 800 can be configured to inject current between an electrode on one or more of the first, second, third, or fourth leads 820, 825, 830, 835 and the CAN 801, and to sense the resultant voltage between the same or different electrodes and the CAN 801.

The implantable medical device 800 can integrate one or more other physiologic sensors to sense one or more other physiologic signals, such as one or more of heart rate, heart rate variability, intrathoracic impedance, intracardiac impedance, arterial pressure, pulmonary artery pressure, RV pressure, LV coronary pressure, coronary blood temperature, blood oxygen saturation, one or more heart sounds, physical activity or exertion level, physiologic response to activity, posture, respiration, body weight, or body temperature. The arrangement and functions of these leads and electrodes are described above by way of example and not by way of limitation. Depending on the need of the patient and the capability of the implantable device, other arrangements and uses of these leads and electrodes are.

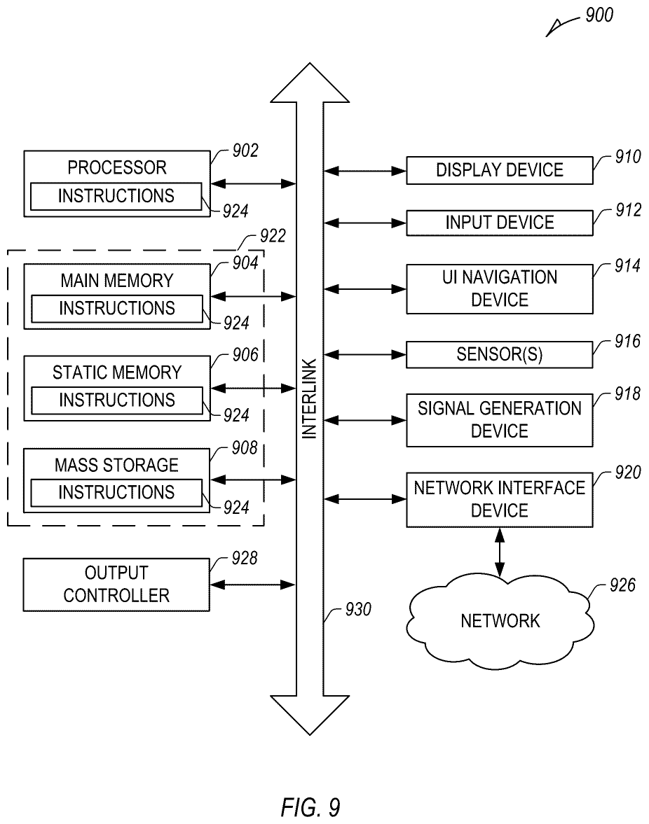

FIG. 9 illustrates a block diagram of an example machine 900 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 900. Circuitry (e.g., processing circuitry, an assessment circuit, etc.) is a collection of circuits implemented in tangible entities of the machine 900 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to perform a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to perform portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 900 follow.

In alternative embodiments, the machine 900 may operate as a standalone device or may be connected (e.g., networked) to other machines. In a networked deployment, the machine 900 may operate in the capacity of a server machine, a client machine, or both in server-client network environments. In an example, the machine 900 may function as a peer machine in peer-to-peer (P2P) (or other distributed) network environment. The machine 900 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 900 (e.g., computer system) may include a hardware processor 902 (e.g., a central processing unit (CPU), a graphics processing unit (GPU), a hardware processor core, or any combination thereof), a main memory 904, a static memory 906 (e.g., memory or storage for firmware, microcode, a basic-input-output (BIOS), unified extensible firmware interface (UEFI), etc.), and mass storage 908 (e.g., hard drive, tape drive, flash storage, or other block devices) some or all of which may communicate with each other via an interlink 930 (e.g., bus). The machine 900 may further include a display unit 910, an alphanumeric input device 912 (e.g., a keyboard), and a user interface (UI) navigation device 914 (e.g., a mouse). In an example, the display unit 910, input device 912, and UI navigation device 914 may be a touch screen display. The machine 900 may additionally include a signal generation device 918 (e.g., a speaker), a network interface device 920, and one or more sensors 916, such as a global positioning system (GPS) sensor, compass, accelerometer, or one or more other sensors. The machine 900 may include an output controller 928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 902, the main memory 904, the static memory 906, or the mass storage 908 may be, or include, a machine-readable medium 922 on which is stored one or more sets of data structures or instructions 924 (e.g., software) embodying or utilized by any one or more of the techniques or functions described herein. The instructions 924 may also reside, completely or at least partially, within any of registers of the processor 902, the main memory 904, the static memory 906, or the mass storage 908 during execution thereof by the machine 900. In an example, one or any combination of the hardware processor 902, the main memory 904, the static memory 906, or the mass storage 908 may constitute the machine-readable medium 922. While the machine-readable medium 922 is illustrated as a single medium, the term “machine-readable medium” may include a single medium or multiple media (e.g., a centralized or distributed database, and/or associated caches and servers) configured to store the one or more instructions 924.

The term “machine-readable medium” may include any medium that is capable of storing, encoding, or carrying instructions for execution by the machine 900 and that cause the machine 900 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 924 may be further transmitted or received over a communications network 926 using a transmission medium via the network interface device 920 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 920 may include one or more physical jacks (e.g., Ethernet, coaxial, or phone jacks) or one or more antennas to connect to the communications network 926. In an example, the network interface device 920 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 900, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A computing device for generating a programming recommendation for an ambulatory medical device to improve cardiac capture in a patient during cardiac resynchronization therapy by the ambulatory medical device, the computing device comprising:
one or more processors; and
one or more memory devices storing instructions, which when executed by the processor, cause the one or more processors to perform operations comprising:
receiving parameter settings of the ambulatory medical device;
processing the received parameter settings by inputting the received parameter settings into one or more pre-trained machine learning models, each of the one or more pre-trained machine learning models trained to compare the received parameter settings to stored model parameter settings from one or more other ambulatory medical devices corresponding to one or more other patients and to identify one or more differences between the parameter settings of the ambulatory medical device and the stored model parameter settings of the one or more other ambulatory medical devices; and
upon obtaining an output from the one or more pre-trained machine learning models indicating the identified one or more differences between the parameter settings of the ambulatory medical device and parameter settings of the one or more other ambulatory medical devices, generating the programming recommendation for the ambulatory medical device to improve cardiac capture for the patient based on the identified one or more differences.

2. The computing device of claim 1, wherein to identify the one or more differences between the parameter settings of the ambulatory medical device and the stored model parameter settings comprises to prioritize the identified one or more differences with respect to reduced cardiac pacing or unsuccessful cardiac capture.

3. The computing device of claim 1, wherein the operations further comprise:
receiving cardiac capture information of the patient during cardiac resynchronization therapy delivered by the ambulatory medical device according to the received parameter settings,
wherein processing the received parameter settings further comprises inputting the received cardiac capture information of the patient into the one or more pre-trained machine learning models, each of the one or more pre-trained machine learning models trained to compare the received parameter settings and the received cardiac capture information to stored model parameter settings and stored model cardiac capture information from one or more other ambulatory medical devices corresponding to one or more other patients and to identify one or more differences between the parameter settings of the ambulatory medical device and the stored model parameter settings of the one or more other ambulatory medical devices,
wherein generating the programming recommendations comprises generating a reprogramming recommendation for the ambulatory medical device to optimize cardiac capture for the patient,
wherein the ambulatory medical device comprises an implantable cardiac resynchronization therapy device implanted in the patient.

4. The computing device of claim 1, wherein the operations further comprise:
receiving physiologic information of the patient obtained by the ambulatory medical device; and
determining an indication of cardiac capture of the patient during cardiac resynchronization therapy delivered by the ambulatory medical device according to the received parameter settings using the received physiologic information.

5. The computing device of claim 1, wherein the operations further comprise:
receiving physiologic information of the patient obtained by the ambulatory medical device,
wherein processing the received parameter settings further comprises inputting the received physiologic information of the patient into the one or more pre-trained machine learning models, each of the one or more pre-trained machine learning models trained to compare the received parameter settings and the received physiologic information of the patient to stored model parameter settings from one or more other ambulatory medical devices corresponding to one or more other patients and stored physiologic information from the one or more other patients and to identify one or more differences between the parameter settings of the ambulatory medical device and the stored model parameter settings of the one or more other ambulatory medical devices,
wherein generating the programming recommendations comprises to optimize cardiac capture for the patient.

6. The computing device of claim 1, wherein the operations further comprise:
receiving information about the patient comprising one of demographic information or medical history information separate from sensed physiologic information of the patient,
wherein processing the received parameter settings further comprises inputting the received information about the patient into the one or more pre-trained machine learning models, each of the one or more pre-trained machine learning models trained to compare the received parameter settings and the received physiologic information of the patient to stored model parameter settings from one or more other ambulatory medical devices corresponding to one or more other patients and stored information about the one or more other patients and to identify one or more differences between the parameter settings of the ambulatory medical device and the stored model parameter settings of the one or more other ambulatory medical devices,
wherein generating the programming recommendations comprises to optimize cardiac capture for the patient.

7. The computing device of claim 1, wherein the operations further comprise:
providing the generated programming recommendation to a user or process.

8. The computing device of claim 7, wherein providing the generated programming recommendation to the user or process includes providing an output of the generated programming recommendation to a user interface for display to the user or to a control circuit to control or adjust the process or function of the ambulatory medical device.

9. The computing device of claim 8, wherein the operations further comprise:
reprogramming the ambulatory medical device using the generated programming recommendation including changes to one or more parameter settings; and
providing cardiac resynchronization therapy to the patient according to the one or more reprogrammed parameter settings.

10. The computing device of claim 1, wherein generating the programming recommendation for the ambulatory medical device to improve cardiac capture for the patient based on the identified one or more differences includes implementing at least one of a set of rules associated with the following parameter settings: Atrioventricular Delay Fixed and Atrioventricular Dynamic Maximum.

11. The computing device of claim 1, wherein generating the programming recommendation for the ambulatory medical device to improve cardiac capture for the patient based on the identified one or more differences includes implementing at least one of a set of rules associated with at least two of the following parameter settings:
Atrial Tachy Response Mode;
Biventricular Trigger Enable;
Ventricular Tachycardia Zone Rate;
Atrial Tachy Response Trigger Rate;
Maximum Sensor Rate Interval;
Ventricular Tachycardia 1 Zone Rate;
Number of Ventricular Zones;
Ventricular Fibrillation Zone Rate;
Atrial Tachy Response Ventricular Rate Regulation Response;
Atrial Tachy Response Biventricular Trigger Enable;
Atrial Tachy Response Lower Rate Limit;
Tachycardia Mode;
Respiration Rate Trend Enable;
Atrial Tachy Response Pacing Chamber; and
Sensing Mode.

12. The computing device of claim 1, wherein generating the programming recommendation for the ambulatory medical device to improve cardiac capture for the patient based on the identified one or more differences includes implementing at least one of a set of rules associated with at least two of the following parameter settings:
Sensed Atrioventricular Delay;
Atrioventricular Dynamic Minimum;
Atrioventricular Delay Fixed; and
Atrioventricular Dynamic Maximum.

13. A computing device for generating a programming recommendation for an ambulatory medical device to improve cardiac capture in a patient during cardiac resynchronization therapy by the ambulatory medical device, the computing device comprising:
one or more processors; and
one or more memory devices storing instructions, which when executed by the processor, cause the one or more processors to perform operations comprising:
receiving physiologic information of the patient obtained by the ambulatory medical device;
receiving parameter settings of the ambulatory medical device;
receiving cardiac capture information of the patient during cardiac resynchronization therapy delivered by the ambulatory medical device according to the received parameter settings;
upon receiving or determining an indication of a loss of cardiac capture of a heart using the received physiologic information of the patient obtained by the ambulatory medical device, processing the received parameter settings by inputting the received parameter settings into one or more pre-trained machine learning models, each of the one or more pre-trained machine learning models trained to compare the received parameter settings to stored model parameter settings from one or more other ambulatory medical devices corresponding to one or more other patients and to identify one or more differences between the parameter settings of the ambulatory medical device and the stored model parameter settings of the one or more other ambulatory medical devices; and
upon obtaining an output from the one or more pre-trained machine learning models indicating the identified one or more differences between the parameter settings of the ambulatory medical device and parameter settings of the one or more other ambulatory medical devices, generating the programming recommendation for the ambulatory medical device based on the identified one or more differences.

14. The computing device of claim 13, wherein the operations further comprise:
upon obtaining an output from the one or more pre-trained machine learning models indicating differences between the parameter settings of the ambulatory medical device and parameter settings of the one or more other ambulatory medical devices, prioritizing the one or more differences with respect to a potential or detected loss of cardiac capture or reduced pacing.

15. A method for generating a programming recommendation for an ambulatory medical device to improve cardiac capture in a patient during cardiac resynchronization therapy by the ambulatory medical device, the method comprising:
receiving, over a network, parameter settings of the ambulatory medical device;
processing, using one or more processors, the received parameter settings by inputting the received parameter settings into one or more pre-trained machine learning models, each of the one or more pre-trained machine learning models trained to compare the received parameter settings to stored model parameter settings from one or more other ambulatory medical devices corresponding to one or more other patients and to identify one or more differences between the parameter settings of the ambulatory medical device and the stored model parameter settings of the one or more other ambulatory medical devices; and
upon obtaining an output from the one or more pre-trained machine learning models indicating the identified one or more differences between the parameter settings of the ambulatory medical device and parameter settings of the one or more other ambulatory medical devices, generating, using the one or more processors, the programming recommendation for the ambulatory medical device to improve cardiac capture for the patient based on the identified one or more differences.

16. The method of claim 15, wherein to identify the one or more differences between the parameter settings of the ambulatory medical device and the stored model parameter settings comprises to prioritize the identified one or more differences with respect to reduced cardiac pacing or unsuccessful cardiac capture.

17. The method of claim 15, comprising:
receiving, over the network, cardiac capture information of the patient during cardiac resynchronization therapy delivered by the ambulatory medical device according to the received parameter settings,
wherein processing the received parameter settings further comprises inputting the received cardiac capture information of the patient into the one or more pre-trained machine learning models, each of the one or more pre-trained machine learning models trained to compare the received parameter settings and the received cardiac capture information to stored model parameter settings and stored model cardiac capture information from one or more other ambulatory medical devices corresponding to one or more other patients and to identify one or more differences between the parameter settings of the ambulatory medical device and the stored model parameter settings of the one or more other ambulatory medical devices,
wherein generating the programming recommendations comprises generating a reprogramming recommendation for the ambulatory medical device to optimize cardiac capture for the patient,
wherein the ambulatory medical device comprises an implantable cardiac resynchronization therapy device implanted in the patient.

18. The method of claim 15, comprising:
receiving, over the network, physiologic information of the patient obtained by the ambulatory medical device; and
determining, using the one or more processors, an indication of cardiac capture of the patient during cardiac resynchronization therapy delivered by the ambulatory medical device according to the received parameter settings using the received physiologic information.

19. The method of claim 15, comprising:
receiving, over the network, physiologic information of the patient obtained by the ambulatory medical device,
wherein processing the received parameter settings further comprises inputting the received physiologic information of the patient into the one or more pre-trained machine learning models, each of the one or more pre-trained machine learning models trained to compare the received parameter settings and the received physiologic information of the patient to stored model parameter settings from one or more other ambulatory medical devices corresponding to one or more other patients and stored physiologic information from the one or more other patients and to identify one or more differences between the parameter settings of the ambulatory medical device and the stored model parameter settings of the one or more other ambulatory medical devices,
wherein generating the programming recommendations comprises to optimize cardiac capture for the patient.

20. The method of claim 15, comprising:
receiving information about the patient comprising one of demographic information or medical history information separate from sensed physiologic information of the patient,
wherein processing the received parameter settings further comprises inputting the received information about the patient into the one or more pre-trained machine learning models, each of the one or more pre-trained machine learning models trained to compare the received parameter settings and the received physiologic information of the patient to stored model parameter settings from one or more other ambulatory medical devices corresponding to one or more other patients and stored information about the one or more other patients and to identify one or more differences between the parameter settings of the ambulatory medical device and the stored model parameter settings of the one or more other ambulatory medical devices,
wherein generating the programming recommendations comprises to optimize cardiac capture for the patient.
